(19) United States
(12) Patent Application Publication
(10) Pub. No.: US 2004/0094155 A1
(43) Pub. Date: May 20, 2004

(54) Arrangement for passive gas sampling

(75) Inventors: Rolf Castor, Hagersten (SE); Magnus Hallback, Bromma (SE)

Correspondence Address: SCHIFF HARDIN & WAITE, Patent Department, 6600 Sears Tower, 233 South Wacker Drive, Chicago, IL, 60606, US

(73) Assignee: Siemens Elema AB

(21) Appl. No.: 10/687,312

(22) Filed: Oct. 16, 2003

(30) Foreign Application Priority Data
Nov. 20, 2002 (SE) 0203428-8

(51) Int. Cl. A61M 16/00

(52) U.S. Cl. 128/204.22

(57) ABSTRACT

An arrangement for passive gas sampling in a breathing system has a conduit having a venturi-nozzle for passively generating pressure variations in the conduit dependent on a flow of breathing gas in the conduit, and a first port and a second port terminating in the conduit and connected to a measurement chamber. The first port and the second port are arranged relative to the venturi-nozzle such that the pressure variations force a flow of breathing gas through the first port into the measurement chamber and out through the second port.

### BACKGROUND OF THE INVENTION

1. Field of the Invention

The present invention relates to an arrangement for passive gas sampling of the type suitable for sampling a breathing gas.

2. Description of the Prior Art

In breathing systems such as ventilators and anesthetic apparatuses the breathing gas is analyzed regularly. This may be done directly in the main supply (with a so-called mainstream-analyzer) or by diverting a gas sample to a measuring chamber (so-called sidestream-analyzer).

The diversion of the gas sample can be done actively by means of a pump or the like or passively, for example by creating a pressure variation between the pressure chamber's inlet and outlet. An example of the latter is described in U.S. Pat. No. 6,450,968.

### SUMMARY OF THE INVENTION

An object of the present invention is to provide an alternative to known arrangements for passive gas sampling.

The above object is achieved in accordance with the present invention in an arrangement for passive gas sampling in a breathing system, having a conduit with venturi-nozzle for passively generating pressure variations in the conduit dependent on a flow of breathing gas in the conduit, a measurement chamber, a first port, and a second port terminating in the conduit and connected to the measurement chamber. The first port and the second port are disposed relative to the venturi-nozzle so that the pressure variations force a flow of the breathing gas through the first port into the measurement chamber and out through the second port.

Pressure differences along a relatively short length can be generated with a venturi-nozzle in a conduit. This pressure difference can be employed to passively conduct a gas sample through a first port to a measurement chamber. Simultaneously, an earlier gas sample can be conducted via a second port from the measurement chamber back to the conduit.

### DESCRIPTION OF THE DRAWINGS

FIG. 1 shows a first embodiment of an arrangement according to the invention.

FIG. 2 shows a second embodiment of an arrangement according to the invention.

### DESCRIPTION OF THE PREFERRED EMBODIMENTS

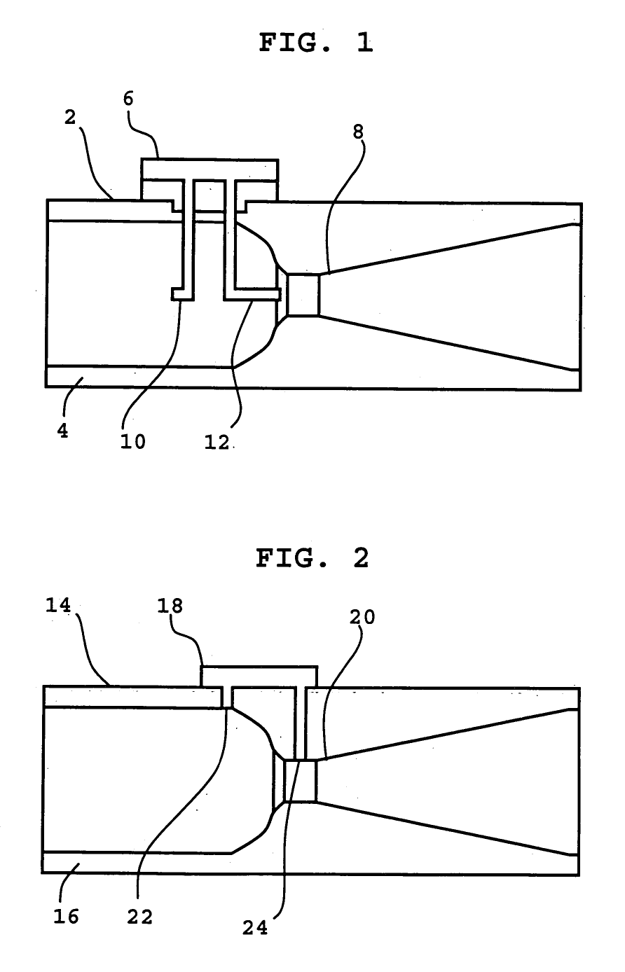

A first embodiment of an arrangement 2 according to the invention is shown in FIG. 1. The arrangement 2 has a body element with a conduit 4 therethrough that a breathing gas can flow (it is irrelevant to the invention whether it is an inspiration flow or an expiration flow). A gas sample can be passively transferred from the breathing gas flow to a measurement chamber 6 for analysis. The analysis can be achieved in any known manner, for example optically, acoustically or electrochemically.

In order to achieve a passive sampling-flow to the measurement chamber 6, a venturi-nozzle 8 is arranged in the conduit 4. A first port 10 and a second port 12, both connected to the measurement chamber 6, are arranged in the conduit 4 so that the pressure variation produced by the venturi-nozzle 8 can be optimally utilized to obtain the sampling-flow into the measurement chamber 6 through the first port 10. An earlier sample will, at the same time, be conducted out of the measurement chamber 6 through the second port 12 and back to the breathing gas flow in the conduit 4.

The gas sample in the measurement chamber 6 will, in principle, be exchanged once every breathing cycle, during inspiration or expiration. The analysis in the measurement chamber 6 may be done continuously when a new gas sample flows into the measurement chamber 6 or during the pause that is created when a change of sample does not occur (expiration or inspiration).

A variation is shown in FIG. 2 wherein an arrangement 14 according to the invention has a conduit 16, a measurement chamber 18, a venturi-nozzle 20, a first port 22 and a second port 24.

The difference from the first embodiment is, in principle, that in the arrangement 14 according to the second exemplary embodiment the ports 22, 24 have holes in the tube 16 that are strategically located to utilize the pressure variation generated by the venturi-nozzle.

Although modifications and changes may be suggested by those skilled in the art, it is the intention of the inventor to embody within the patent warranted hereon all changes and modifications as reasonably and properly come within the scope of his contribution to the art.

We claim as our invention:

1. An arrangement for passive gas sampling in a breathing system, comprising:
a body element having a conduit therein adapted to receive a flow of breathing gas;
a measurement chamber associated with said body element;
a venturi-nozzle in said conduit for passively generating pressure variations in said conduit dependent on said flow of breathing gas in said conduit;
said body element having a first port and a second port each terminating in said conduit and each connected to said measurement chamber; and
said first port and said second port being disposed relative to said venturi-nozzle so that said pressure variations force a flow of said breathing gas through said first port into said measurement chamber and out through said second port.
